(19) United States
(12) Patent Application Publication
(10) Pub. No.: US 2025/0180405 A1
(43) Pub. Date: Jun. 5, 2025

(54) SYSTEM AND METHOD FOR IMAGING HISTOPATHOLOGY-STAINED SLIDES USING COHERENT ANTI-STOKES RAMAN SPECTRAL IMAGING

(71) Applicant: QUISITUM AUTOMATED SYSTEMS INC., Vancouver (CA)

(72) Inventors: Haishan ZENG, Vancouver (CA); Kornelia Oswald DIESEL, Hohenpeißenberg (DE); Calum MACAULAY, Vancouver (CA); Zhenguo WU, Richmond (CA); Branko Mihael PALCIC, Whistler (CA)

(21) Appl. No.: 18/842,964

(22) PCT Filed: Mar. 3, 2023
(86) PCT No.: PCT/CA2023/050285

§ 371 Date: Aug. 30, 2024

Related U.S. Application Data
(60) Provisional application No. 63/315,981, filed on Mar. 3, 2022.

(51) Int. Cl. G01J 3/44; G01J 3/02; G01N 33/483

(52) U.S. Cl. CPC G01J 3/4412; G01J 3/0248; G01J 3/0264; G01J 3/4406; G01N 33/4833

(57) ABSTRACT

A system for multimodal imaging of histology-stained slides comprises a white light imaging system for providing a white light image of a field of view of a tissue sample on a slide. A user interface receives user input specifying a region of interest (ROI). A Raman spectroscopy system is configured to perform Raman spectroscopy on the ROI. A display is connected to display a Raman spectrogram and/or an image of the ROI acquired by the Raman spectroscopy system. The Raman spectroscopy system may perform coherent anti-stokes Raman scattering (CARS) imaging of the histology-stained slides. The system and related methods have application in efficient workflows for pathology reviews of histology stained slides augmented by Raman spectroscopy information.

### CROSS-REFERENCE TO RELATED APPLICATIONS

This application claims priority from U.S. application No. 63/315,981 filed 3 Mar. 2022 and entitled SYSTEM AND METHOD FOR IMAGING HISTOPATHOLOGY-STAINED SLIDES USING COHERENT ANTI-STOKES RAMAN SPECTRAL IMAGING which is hereby incorporated herein by reference for all purposes. For purposes of the United States of America, this application claims the benefit under 35 U.S.C. § 119 of U.S. application No. 63/315,981 filed 3 Mar. 2022 and entitled SYSTEM AND METHOD FOR IMAGING HISTOPATHOLOGY-STAINED SLIDES USING COHERENT ANTI-STOKES RAMAN SPECTRAL IMAGING which is hereby incorporated herein by reference for all purposes.

### FIELD

The present disclosure generally relates to coherent anti-stokes Raman scattering (CARS) imaging and more particularly to a coherent anti-stokes Raman scattering (CARS) imaging of histology-stained slides.

### BACKGROUND

Histopathology is the current “gold standard” for many disease diagnoses. General histopathology assessment is based on visual examination of slides that are stained with hematoxylin and eosin (“H&E”). H&E stains cell cytoplasm, nuclei, and extracellular matrix components to provide mainly morphology information. A pathologist may use this information in combination with their expertise to differentiate diseases/cancers from benign lesions or normal tissues. However, morphology information alone is often insufficient as a basis for a confirmative diagnosis.

Molecular information, for example, the presence or absence of specific cellular proteins (detected by antibody labeling for example) and the presence or absence of specific DNA alterations (measured by some form of DNA sequencing or labeled complementary DNA strands) can be used together with morphology information to provide more reliable diagnoses. Knowledge of the molecular status is becoming more and more important for precision medicine with respect to both diagnosis and treatment.

Raman spectroscopy, based on the detection of Raman scattered light, is a well-known method for retrieving information regarding molecules present in tissue in vivo or ex vivo. However, because of the weak excitation efficiency of Raman scattering, it can take a relatively long time (on the order of seconds) to acquire a single Raman spectrum. The problem that Raman spectra take an undesirably long time to acquire becomes worse if high spatial resolution is required as in the case of histopathology analysis where Raman spectra for a large number of target points need to be acquired.

Coherent anti-stokes Raman scattering (CARS) is a type of enhanced Raman scattering. CARS greatly reduces the time needed to acquire biochemical information from tissue. By using CARS imaging it is possible to determine the spatial distribution of specific molecules over a field of view in a reasonably short period of time.

Several methods for CARS imaging have been developed. These include: narrowband CARS [9]; multiplex broadband CARS [10, 11]; and three-color excited broadband CARS. Narrowband CARS uses two narrow-band picosecond lasers as the pump and Stokes beams to acquire a single Raman band. Through wavelength shifting of either the pump beam or the Stokes beam, multiple Raman bands can be acquired in the fingerprint region. Multiplex CARS allows direct acquisition of broadband CARS spectra by using a narrow-band laser as the pump beam and a broadband laser as the Stokes beam [10, 12]. The non-resonance background is a challenge for CARS signal detection for narrowband CARS and multiplex CARS [13], especially in the weak fingerprint region. High-speed coherent Raman fingerprint imaging of biological tissues has been reported based on the intrapulse three-color excitation method [14]. The imaging speed of the 3-color excitation method as implemented in [14]. was limited by the data acquisition charge coupled device (CCD) to 3.5 ms per spectrum or 4.5 minutes per image with a pixel resolution of 300 pixels×300 pixels.

There remains a need for systems and methods that may be used to acquire information to support diagnoses of health issues such as cancer. There is a particular need for such methods and systems that are compatible with existing histopathology workflows.

### SUMMARY

The present invention has a number of aspects. These include without limitation:
methods and systems for reviewing stained pathology slides; and
methods and systems for combined white-light imaging and at least one of CARS imaging and confocal Raman spectroscopy of regions of interest in stained pathology slides.
   The pathology slides may, for example, be H&E slides, slides stained with Papanicolaou staining, or slides with Masson's Trichrome staining.

One example aspect of the invention provides systeme for multimodal imaging of histology-stained slides. The systems comprise a white light imaging system configured to provide a white light image of a field of view of a tissue sample on the histology-stained slide and a user interface operable to receive user input specifying a region of interest (ROI) on the tissue sample. The systems also include a Raman spectroscopy system. The Raman spectroscopy system comprises a light source operable to generate an excitation beam, a scanner operable to scan the excitation beam over the ROI, and a spectrometer coupled to receive Raman scattered light resulting from interaction of the excitation beam and the tissue sample. The Raman spectroscopy system is configured to perform Raman spectroscopy on the ROI to obtain Raman spectra. A display is connected to display Raman spectroscopy information acquired by the Raman spectroscopy system. The a Raman spectroscopy information may, for example comprise a Raman spectrogram and/or an image of the ROI acquired by the Raman spectroscopy system.

In some embodiments the system is configured to display the Raman spectrogram and/or an image of the ROI acquired by the Raman spectroscopy system superposed on a portion of the white light image.

In some embodiments the Raman spectroscopy system comprises a coherent anti-Stokes Raman scattering (CARS) system configured to generate a hyperspectral or multispectral Raman image of at least the ROI. In some embodiments the spectrometer is integrated with the CARS system. In some embodiments the light source of the Raman spectroscopy system comprises a pulsed laser

In some embodiments the light source of the Raman spectroscopy system comprises a pulsed narrowband probe laser and a pulsed broadband laser. In some embodiments the system comprises a delay line arranged to apply a controllable delay to light from the probe laser. In some embodiments the system comprises a beamsplitter arranged to combine the light from the probe laser and the broadband laser to yield the excitation beam.

In some embodiments the spectrometer comprises a light sensing array comprising an array of high sensitivity light detectors, for example, photomultiplier tubes (PMTs) or single-photon avalanche photodiodes (SPADs). The light sensing array may, for example, include at least five of the PMTs or SPADs. In some embodiments the light sensing array includes 24 to 500 of the PMTs or SPADs.

In some embodiments the spectrometer is a virtual slit spectrometer. In some such embodiments the scanner causes the Raman scattered light to be scanned back and forth along the virtual slit of the spectrometer.

In some embodiments the Raman spectroscopy system is arranged to receive the Raman scattered light that is emitted in a forward direction on a side of the histology stained slide opposite to a side of the histology stained slide on which the excitation beam is incident. The scanner may, for example, comprise a first rotating polygonal mirror. In some embodiments the Raman scattered light is directed to be reflected from the first rotating polygonal mirror and to be descanned by the first rotating polygonal mirror. In some embodiments the system comprises a second rotating polygonal mirror and the Raman scattered light is directed to be reflected from the second rotating polygonal mirror and to be descanned by the second rotating polygonal mirror.

In some embodiments the Raman spectroscopy system is arranged to receive the Raman scattered light that is emitted in a backward direction on a side of the histology stained slide that is the same as a side of the histology stained slide on which the excitation beam is incident. In some embodiments the Raman scattered light is guided to pass through the scanner and to be descanned by the scanner.

In some embodiments the system is configured to collect: a forward component of the Raman scattered light that is emitted in a forward direction on a side of the histology stained slide opposite to a side of the histology stained slide on which the excitation beam is incident, and a backward component of the Raman scattered light that is emitted on a side of the histology stained slide that is the same as a side of the histology stained slide on which the excitation beam is incident, to combine the forward and backward components of the Raman scattered light and to direct the combined forward and backward components of the Raman scattered light into the spectrometer. In some such embodiments the system comprises an optical reflector arranged to redirect one of the forward and backward components of the Raman scattered light to pass though the histology stained slide before reaching the spectrometer.

In some embodiments the system comprises a controller configured to obtain spectra from the spectrometer in coordination with operation of the scanner and to associate each of the spectra with a location within the ROI. In some embodiments the controller comprises a data store containing process information that characterizes Raman spectra of processing chemicals and/or stains and the controller is configured to identify one or more components of the obtained spectra of the Raman scattered light that correspond to the processing chemicals and/or stains based on the process information. In such embodiments the controller may be configured to remove the components of the obtained spectra of the Raman scattered light that correspond to the processing chemicals and/or stains.

In some embodiments the tissue sample comprises a first part and a second part, the first part of the tissue sample being on the histology stained slide and the second part of the tissue sample being on a second slide wherein the white light imaging system is configured to image the first part of the tissue sample on the histology stained slide, the Raman spectroscopy system is configured to obtain the Raman spectrogram and/or image from the second part of the tissue sample and the controller is configured to create a mapping that indicates the locations of corresponding features that are shown in both of the first and second parts of the tissue sample. In some embodiments the histology stained slide and the second slide are mounted to the same stage such that movements of the stage move the histology stained slide and the second slide together. In some embodiments the scanner comprises a mechanism for moving the stage. In some embodiments the mapping comprises a transformation comprising a rotation and/or a translation which takes a point on the first part of the tissue sample to a corresponding point on the second part of the tissue sample. In some embodiments the controller is configured to generate the mapping by processing low magnification white light images of the first and second parts of the tissue sample to locate coordinate pairs that indicate positions of structures present in both of the first and second parts of the tissue sample.

In some embodiments the user interface comprises a pointing device operable to specify the ROI by drawing a boundary of the ROI on the displayed white light image. The pointing device may, for example, comprise a stylus, a cursor having a position controlled by a user input such as a mouse, trackball, directional keys, a touch screen and/or the like.

In some embodiments the Raman spectroscopy system comprises a scanning unit that is operable to provide scanning on first and second axes wherein the scanning on the first axis is faster than the scanning on the second axis. In some embodiments the scanning unit comprises one or more of: a galvo scanner, a resonance scanner, an opto-acoustic scanner, an actuator-controlled tilting mirror (e.g. controlled by a piezoelectric actuator) and a rotating polygonal mirror scanner. In some embodiments the scanning unit comprises a mechanism operative to move a stage that supports the histology stained slide. In some embodiments the scanning unit employs separate scanners for two dimensions (e.g. X and Y dimensions). In such embodiments the separate scanners may comprise scanning mechanisms of different types.

In some embodiments the system is configured to perform imaging of the tissue sample using one or more additional imaging modality. The additional imaging modality may, for example be selected from the group consisting of: reflectance confocal imaging; two-photon fluorescence imaging; and second harmonic generation imaging. In some embodiments the additional imaging modality may be performed without demounting the histology stained slide from a stage on which the histology stained slide is mounted. In some embodiments images obtained by the alternative imaging modality are registered with the white light image and/or an image based on information obtained by the Raman spectrometry system. In some embodiments the system is configurable to selectively perform imaging using the alternative imaging modality on the ROI.

Another example aspect of the invention provides methods for multimodal imaging of histology-stained slides. The methods comprise imaging a histology stained slide with a white light imaging system to provide a white light image of a field of view of a tissue sample on the histology-stained slide and displaying the white light image to a user as well as receiving by a user interface user input specifying a region of interest (ROI) on the tissue sample. Based on the user input, a Raman spectroscopy system is operated to obtain Raman spectra for a portion of the tissue sample corresponding to the ROI. Information derived from the Raman spectroscopy system (e.g. a Raman spectrogram and/or an image of the ROI acquired by the Raman spectroscopy system) is displayed.

In some embodiments the histology stained slide is a H&E stained slide, a slide carrying a tissue sample that is stained with Papanicolaou staining, or a slide carrying a tissue sample stained with Masson's Trichrome staining.

In some embodiments the method comprises displaying the Raman spectrogram and/or an image of the ROI acquired by the Raman spectroscopy system superposed on a portion of the white light image.

In some embodiments obtaining the Raman spectra comprises obtaining a coherent anti-Stokes Raman scattering (CARS) hyperspectral or multispectral Raman image of at least the ROI.

In some embodiments obtaining the Raman spectra comprises detecting spectral components of the Raman scattered light at an array comprising an array of sensitive light detectors such as photomultiplier tubes (PMTs) or single-photon avalanche photodiodes (SPADs). In some embodiments the light sensing array includes at least five of the PMTs or SPADs. For example, the light sensing array may include 24 to 500 of the PMTs or SPADs.

In some embodiments the method comprises scanning the Raman scattered light back and forth along a virtual slit of a virtual slit spectrometer.

In some embodiments the method comprises descanning the Raman scattered light.

In some embodiments the method comprises collecting: a forward component of the Raman scattered light that is emitted in a forward direction on a side of the histology stained slide opposite to a side of the histology stained slide on which the excitation beam is incident, and a backward component of the Raman scattered light that is emitted on a side of the histology stained slide that is the same as a side of the histology stained slide on which the excitation beam is incident, and combining the forward and backward components of the Raman scattered light.

In some embodiments the method comprises obtaining a plurality of the Raman spectra and associating each of the Rama spectra with a corresponding location within the ROI.

In some embodiments, the method comprises identifying one or more components of the obtained spectra of the Raman scattered light that correspond to processing chemicals and/or stains present in the histology stained slide.

In some embodiments the method comprises removing the components of the obtained spectra of the Raman scattered light that correspond to the processing chemicals and/or stains.

In some embodiments the tissue sample comprises a first part and a second part, the first part of the tissue sample being on the histology stained slide and the second part of the tissue sample being on a second slide wherein the white light imaging images the first part of the tissue sample on the histology stained slide, and the Raman spectra are obtained from the second part of the tissue sample. In some embodiments the method comprises creating a mapping that indicates the locations of corresponding features that are shown in both of the first and second parts of the tissue sample. In some embodiments the mapping comprises a transformation comprising a rotation and/or a translation which takes a point on the first part of the tissue sample to a corresponding point on the second part of the tissue sample. In some embodiments generating the mapping comprises processing low magnification white light images of the first and second parts of the tissue sample to locate coordinate pairs that indicate positions of structures present in both of the first and second parts of the tissue sample.

In some embodiments specifying the ROI comprises drawing a boundary of the ROI on the white light image.

In some embodiments the method comprises, without removing the histology stained slide from an apparatus comprising the white light imaging system and the Raman spectroscopy system, imaging the tissue sample using one or more additional imaging modality, the additional imaging modality selected from the group consisting of: reflectance confocal imaging; two-photon fluorescence imaging; and second harmonic generation imaging.

Another example aspect of the invention provides methods for multimodal imaging of an object on a histology-stained slide. The methods comprise: obtaining a white light image of a histology-stained slide; selecting at least one region of interest (ROI) in the object; generating a hyperspectral or multispectral Raman image of the at least one ROI in the object; and processing the hyperspectral or multispectral Raman image to obtain biochemical information regarding the object.

The above aspects may be applied to facilitate review of histology trained slides by a person, such as a pathologist, wherein the review is enhanced by the availability of information derived from a Raman spectroscopy system without disrupting a workflow for review of the histology stained slides. In some embodiments the Raman spectroscopy information is obtained sufficiently rapidly that the review process is not significantly delayed by acquisition of the Raman spectroscopy information.

Another example aspect of the invention provides apparatus having any new and inventive feature, combination of features, or sub-combination of features as described herein.

Another example aspect of the invention provides methods having any new and inventive steps, acts, combination of steps and/or acts or sub-combination of steps and/or acts as described herein.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

Throughout the drawings, reference numbers may be re-used to indicate correspondence between referenced elements. The drawings are provided to illustrate example embodiments described herein and are not intended to limit the scope of the disclosure. Sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 is a schematic view showing stages of an example process for direct CARS imaging of a selected region of interest (ROI) in a histology-stained slide.

FIGS. 2-12 are schematic view of example systems for CARS imaging and/or confocal Raman spectroscopy of histology-stained slides according to various example embodiments of the invention.

FIG. 13 is a schematic view showing stages of an example process for CARS imaging and confocal Raman spectroscopy of a preselected ROI on an unstained slide of an adjacent tissue section.

FIGS. 14A to 14G are schematic illustration which accompany a description of stages of an example algorithm for image tracing.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

The present technology provides systems and methods for direct CARS imaging and/or confocal Raman spectroscopy of stained pathology slides. The slides may, for example, be H&E slides, slides with Papanicolaou staining, or slides with Masson's Trichrome staining. The technology may be implemented in ways that seamlessly incorporate a CARS modality into histopathology workflows. Some embodiments allow pathologists to examine stained slides as usual initially. The pathologists can then identify any suspicious regions in the field of view (e.g. by outlining the suspicious regions). CARS imaging may then be performed on the identified suspicious regions. The CARS imaging process can generate hyperspectral images of the identified suspicious regions that are co-registered with an image of a stained slide. The co-registered CARS images can provide rich biochemical information for a more accurate diagnosis. Other imaging channels such as one or more of: reflectance confocal imaging, two-photon fluorescence imaging, and second harmonic generation imaging may optionally be integrated with the same imaging system to give more comprehensive information.

A problem is that stained slides generally include traces of the chemicals used in preparing the slides. These chemicals can influence Raman/CARS spectra. These chemicals typically include formaldehyde for tissue fixation, paraffin wax for tissue embedding, xylene for tissue clearance and dewaxing, and stains. The following discussion explains application of the present technology to H&E stained slides. Those of skill in the art will understand from this description that the technology may be applied mutatis mutandis to slides stained with other selections of stains.

Formaldehyde has been reported to have weak influence on tissue Raman spectra by several research groups [1-3]. Paraffin wax is a typical embedding material, which has strong Raman peaks and can cause serious contamination if not removed from the tissue before the staining step[4-6]. In existing workflows for preparing H&E slides, dewaxing is performed before staining. An optimized dewaxing method that includes treating the paraffin-embedded tissue in xylene for more than 30 minutes has been reported to minimize the effect of paraffin wax on Raman spectra [5]. Xylene can be removed from the tissue in dehydration steps (which may involve placing the tissue in 50, 70, and 100% ethanol for 5 min [5]). After H&E staining, according to the standard protocol, the tissue will be further dehydrated and cleared with ethanol followed by xylene as the last step. [https://www.leicabiosystems.com/knowledge-pathway/he-staining-overview-a-guide-to-best-practices/]. The effects of xylene and H&E stains in an H&E prepared slide on Raman signals of tissue in the slides has not been well studied. Two groups have reported identifying specific Raman bands of H&E stained tissue despite the presence of H&E staining agents [7, 8].

Potential problems caused by the possible presence of processing chemicals in H&E stained slides may be avoided or reduced by:
processing the H&E slides in a way which reduces the presence of processing chemicals such as wax and xylene in the prepared H&E slides; and
acquiring CARS signals over broad ranges such as the full fingerprint range (0 cm⁻¹ to 2000 cm⁻¹) and the high wavenumber range (>2000 cm⁻¹).
   In a broadband CARS signal it is possible to identify and isolate/remove the sharp peaks resulting from residual chemicals used for tissue processing. Useful tissue Raman information that is confidently not contaminated/compromised by the effects of processing and staining may then be obtained. Where broadband CARS Signals are obtained under intrapulse 3-color excitation, the strong non-resonance background tends to be advantageous because it tends to amplify the inherently weak fingerprint signal by heterodyne amplification as described, for example in reference [14].

Some embodiments of the present technology use the three-color excitation method of acquiring CARS signals to directly image H&E stained tissue with the entire biologically relevant Raman window (0-3500 cm⁻¹). In a prototype system designed to demonstrate the present technology a narrow band probe laser and a supercontinuum femtosecond laser were used for CARS imaging.

Imaging speed is important in clinical applications. An imaging technology that is too slow is not likely to be accepted. The inventors have determined that the speed of CARS imaging may be increased by one or more of:
using a spectrometer that uses arrays of fast optical sensors such as photomultiplier tubes (PMTs), avalanche photodiodes (APDs), single-photon avalanche diodes (SPADs), charge coupled detectors (CCDs) and CMOS (complementary metal-oxide semiconductor) detectors to detect light;
using optical scanners to acquire CARS images;
integrating a spectrometer for CARS imaging into an imaging system; and
performing CARS imaging only on areas identified by a user and not performing CARS imaging on other areas that are presumably non-relevant for diagnosis.

CARS imaging may be performed quickly enough to be incorporated into standard workflows for histopathology reviews of H&E slides by incorporating some or all of these features. The present technology provides a practical approach to performing CARS imaging directly on H&E slides as an ancillary technique in clinical pathology practice.

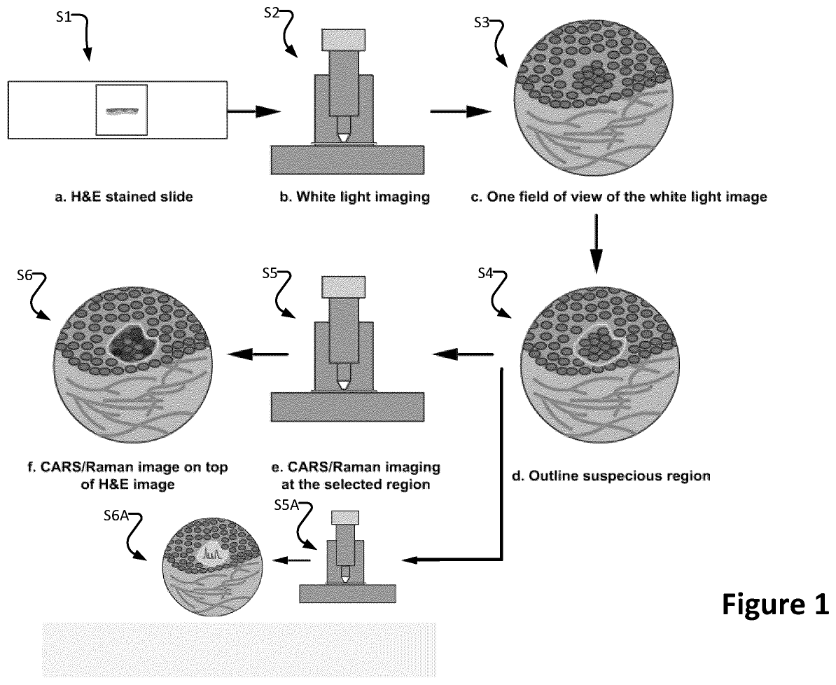

FIG. 1 shows stages in an example method for direct CARS imaging of regions on an H&E stained slide in a clinical pathology workflow. The method may be performed using a microscope imaging system that combines the functions of traditional white light imaging and hyperspectral CARS imaging.

At S1 an H&E stained slide is provided. At S2 white-light imaging is performed to obtain a white light image S3. The white light imaging channel and resulting white light image may be used for “gold standard” visual inspection of the H&E stained slide. The pathologist can also elect to obtain CARS images for any suspicious region(s) in the field of view. For example, FIG. 1 includes identifying a region of interest at S4. For example, the pathologist may use a suitable interface which displays a white light image of the H&E stained slide and receives input defining an outline around a region of interest (ROI) on which CARS hyperspectral imaging will be performed. The input may be provided, for example, using a pointing device such as a stylus, mouse, trackball or fingertip to trace the outline. At S5 CARS imaging is performed. The results of the CARS imaging can include biochemical information about tissues which may assist in making a diagnosis. The biomedical information may be obtained by detecting signatures (e.g. characteristic Raman peaks and/or characteristic patterns of Raman peaks) of various biochemical compounds in the CARS signal as is known to those of skill in the art. At S6 a CARS image is displayed. In some embodiments the CARS image is superposed on the white light image. In addition or in the alternative to S5 and S6, S5A performs confocal Raman spectroscopy on the ROI and S6A displays a Raman spectrum for the ROI. The Raman spectrum may be superposed on the white light image, e.g. as shown, and/or displayed separately.

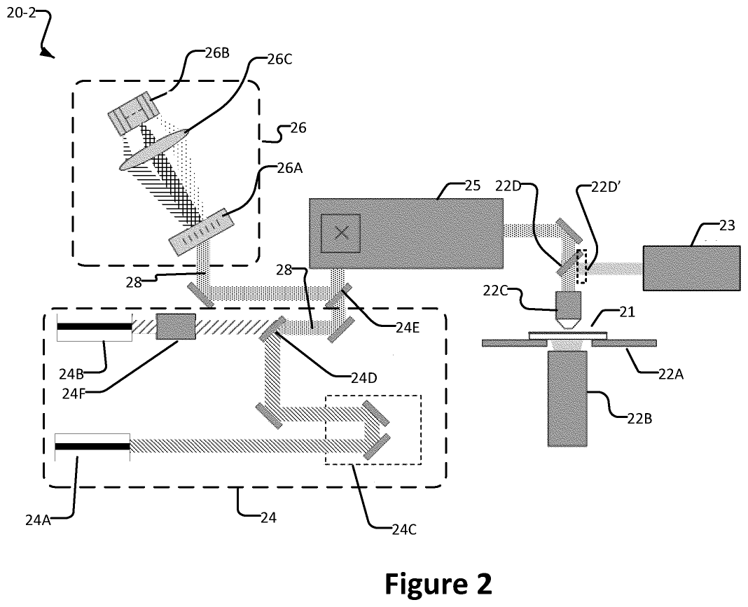

FIG. 2 shows an example microscope imaging system 20 which is operable to selectively perform white light imaging and CARS imaging on an H&E stained slide 21. Slide 21 is supported on a stage 22A. A flip mirror 22D is operable to select between white light and CARS imaging. In FIG. 2 flip mirror 22D is set up for white light imaging. Flip mirror 22D may be moved out of the optical path (e.g. to position 22D′) for CARS imaging.

Light for white light imaging is provided by a trans-illumination unit 22B. A transmission light signal is collected by an objective 22C and imaged at a viewing system 23. Viewing system 23 may include a camera and/or one or more viewing devices such as eyepieces and/or displays that a pathologist may use to observe white light and CARS images of slide 21.

In this example embodiment, CARS signals are obtained by illuminating slide 21 with suitable light and detecting CARS signals that are emitted in a backward direction (i.e. the CARS signals are emitted to the same side of slide 21 from which the light for CARS imaging is incident).

CARS imaging is performed using a CARS light source 24 which provides light to a scanning unit 25 which scans the light from CARS light source 24 over a field of view on slide 21 and also collects a CARS signal in the form of light that is backscattered from the current location of the scanned light. The collected light is delivered to a spectrometer 26 for analysis.

CARS light source 24 comprises a pulsed narrowband probe laser 24A and a pulsed broadband laser 24B. Light from probe laser 24A passes through a delay line 24C to a beamsplitter 24D. Light from broadband laser 24B passes through a pulse compressor 24F and merges with the light from probe laser 24A at beamsplitter 24D. The temporal overlap of pulses of broadband laser light from broadband laser 24B and pulses of light from probe laser 24A is adjustable by altering the amount of delay provided by delay line 24C. The merged beam 28 (which may be called “excitation beam 28”) is then directed to scanning unit 25 which directs the merged laser beam into a back aperture of objective 22C.

Scanning unit 25 is operable to scan the merged laser beam over a field of view on slide 21 by altering an angle at which the merged light is relayed onto the back aperture of objective 22C. Objective 22C focuses the merged laser beam onto a point on the sample.

Scanning unit 25 may be realized in various ways. For example, scanning unit 25 may include:
a resonance scanner operative to scan light in a first direction (e.g. an “X” direction) and a galvo scanner or acousto-optical scanner operative to scan the light in a second direction that is non-parallel to the first direction (e.g. a “Y” direction);
a rotating polygonal mirror operative to scan light in a first direction (e.g. an “X” direction) and a galvo scanner or acousto-optical scanner operative to scan the light in a second direction that is non-parallel to the first direction (e.g. a “Y” direction);
a first galvo scanner or acousto-optical scanner operative to scan light in a first direction (e.g. an “X” direction) and a second galvo scanner or acousto-optical scanner operative to scan the light in a second direction that is non-parallel to the first direction (e.g. a “Y” direction); or
   a two-axis tilting stage (e.g. a stage in which tilt in each of two axes is controlled by fast piezoelectric actuators). In some embodiments scanning unit 25 includes optics that adjust the size of the merged beam to overfill the back aperture of objective 22C.

In some embodiments, scanning unit 25 includes first and second scanners that are each operable to scan light in a respective direction (e.g. X and Y directions) and are each controllable to hold a desired position within a scanning range. Such scanners may be controlled to efficiently collect Raman Spectra for an arbitrary set of points in a field of view and/or for an arbitrarily shaped region of interest.

Interaction of the merged beam with the sample of slide 21 yields Anti-Stokes Raman scattered light (a “CARS signal”). In the embodiment of FIG. 2 the CARS signal is collected and collimated by objective 22C. The CARS signal is descanned by scanning unit 25 and directed to spectrometer 26 by way of a wavelength selective filter 24E which prevents reflected light of the merged beam from light source 24 from reaching spectrometer 26. Filter 24E may, for example comprise a long-pass filter which passes the merged beam of light source 24 but reflects light of shorter wavelengths into spectrometer 26.

In spectrometer 26, the CARS signals are passed through a volume grating. 26A which disperses the CARS signal by wavelength. The dispersed signal is focused onto an array 26B of light detectors (e.g. PMTs or APDs) by a lens 26C. Each of the light detectors is operable to detect photons in a specific wavelength range. The collective outputs of the light directors of array 26B provide a CARS spectrum. Array 26B has a sufficient number of channels to provide a desired wavelength resolution of the CARS spectrum. In some embodiments array 26B has in the range of 8 to 500 channels. For example, array 26B may comprise about 64 channels. Even a few channels (2 to 10 channels) can provide useful biochemical information to augment visual inspection of a white-light image.

The present technology may be varied in a wide range of ways while preserving characteristics that allow CARS information and white light images of H&E stained slides to be presented to a reviewer with minimal interruption to a workflow.

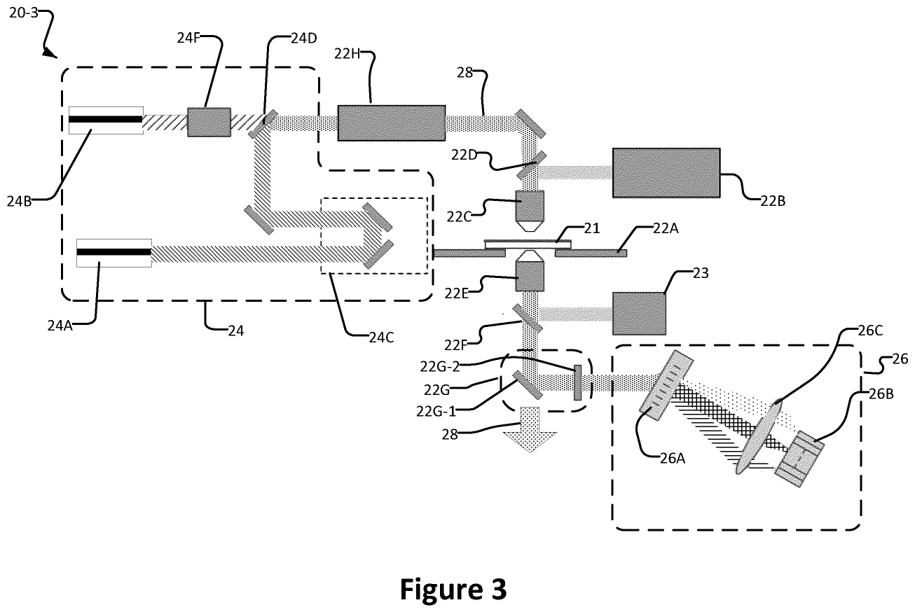

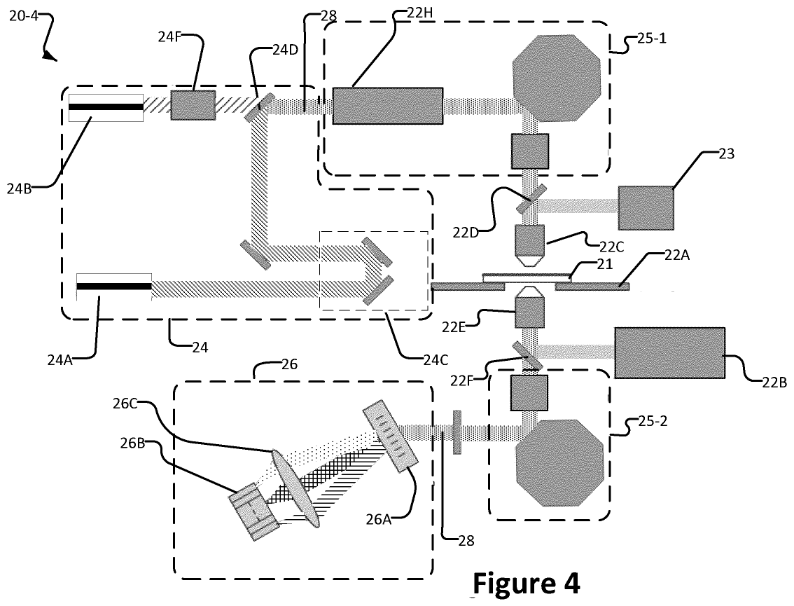

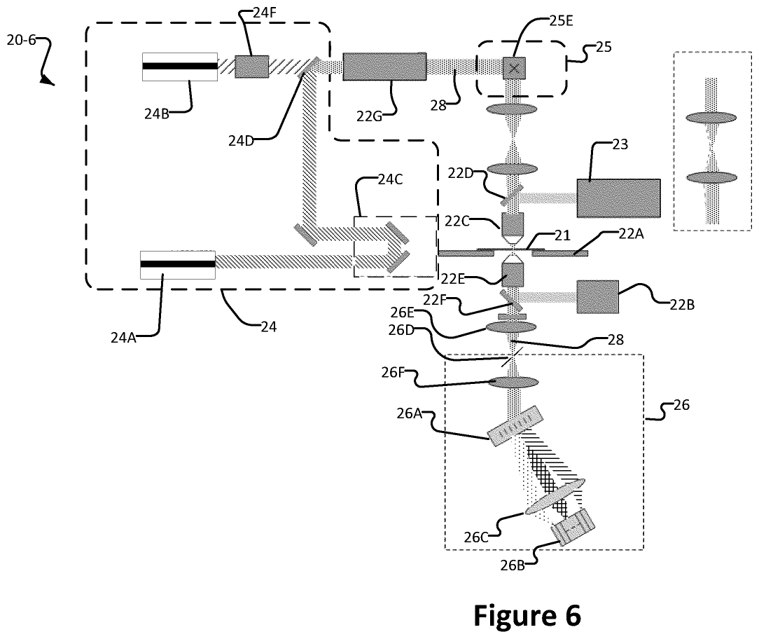

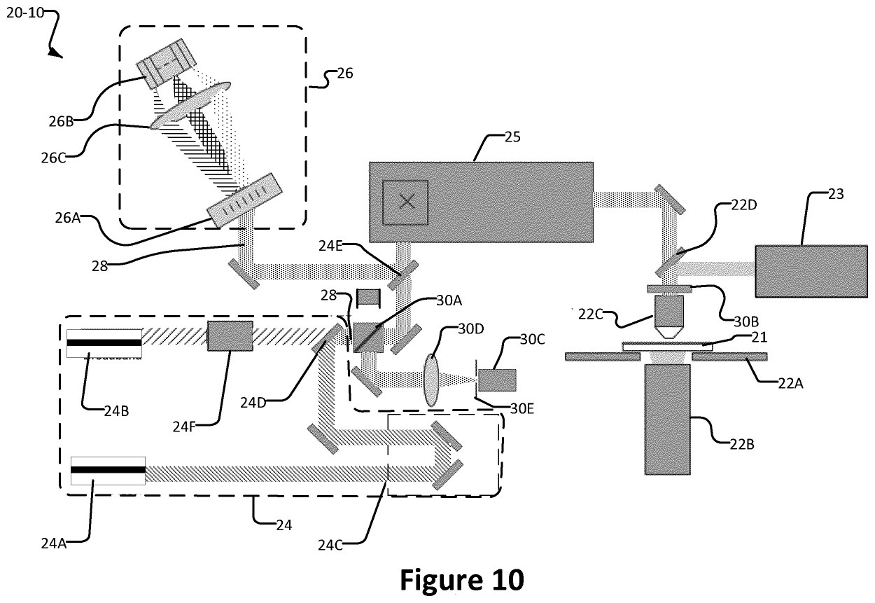

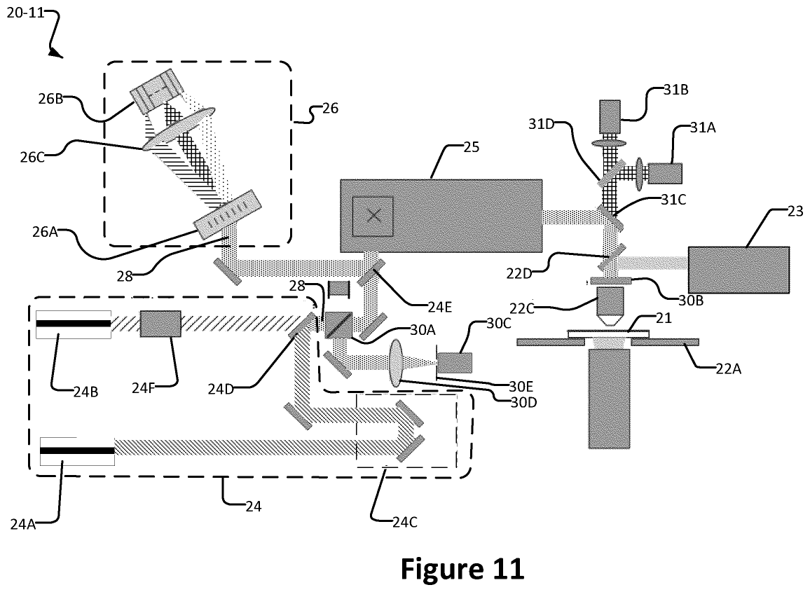

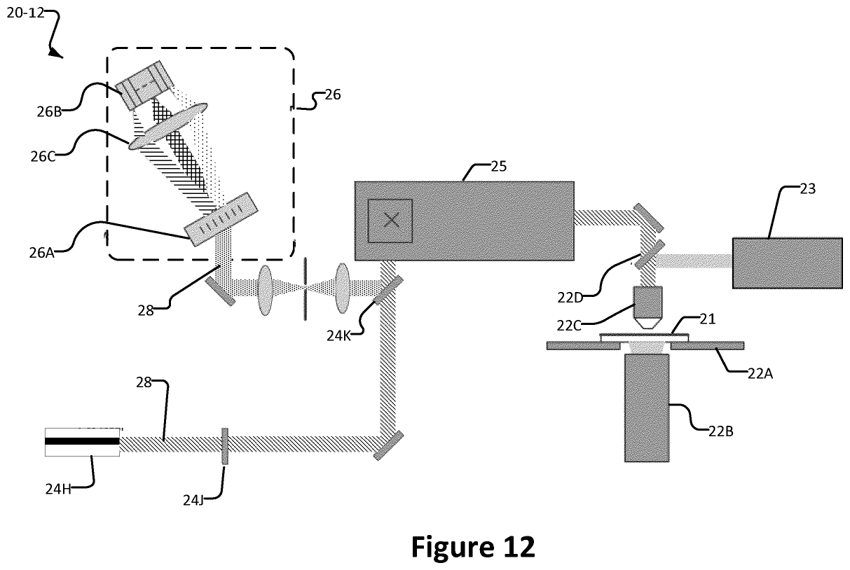

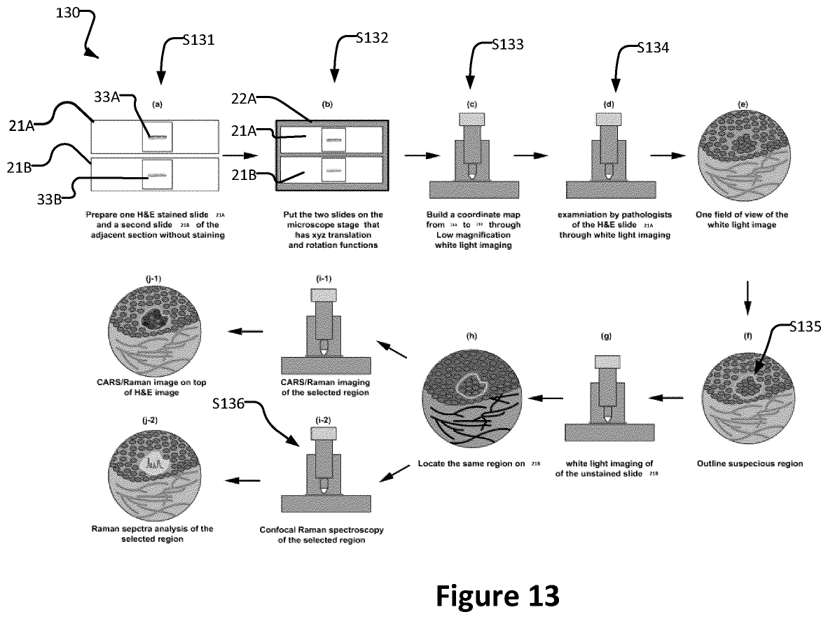

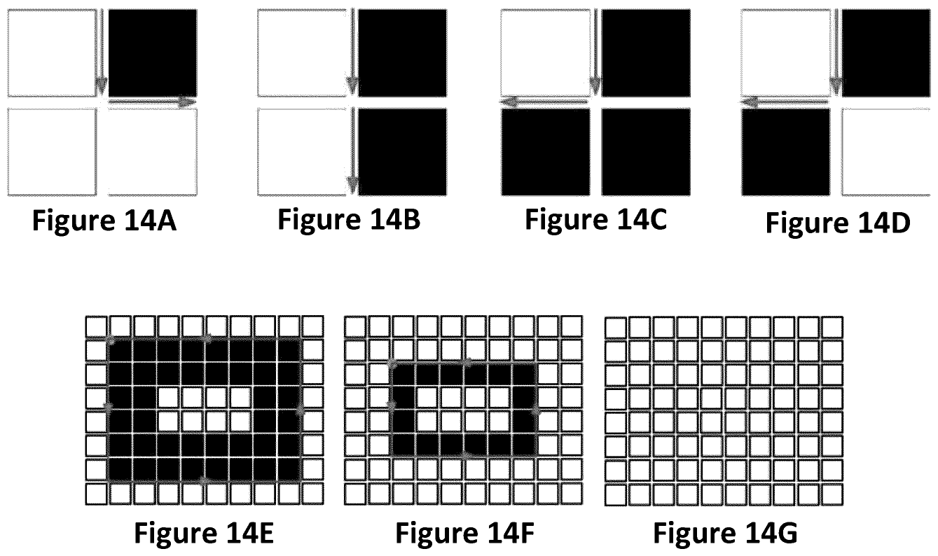

Examples of ways that the technology may be varied include:
a CARS signal may be detected in a “forward” direction (i.e. the CARS signal may be detected on a side of slide 21 opposite to the side of slide 21 on which the merged laser beam (excitation beam 28) is incident—see e.g. FIG. 3 to 9);
light for white light imaging may be incident on slide 21 from the same direction as excitation light for CARS imaging (e.g. as in FIG. 3) or from the opposite direction as the excitation light (e.g. as in FIG. 2);
different arrangements for scanning and descanning in CARS imaging (e.g. as in FIGS. 4, 5, 6, 7, 8, 9)
provisions for additional imaging modes (e.g. as in FIGS. 10, 11)
CARS chemical images are obtained using pulsed laser sources (e.g. as in FIGS. 2 to 11). Equivalent chemical information may also be obtained by conventional Raman spectroscopy using a continuous wave laser source (e.g. as shown in FIG. 12).
The obtained biochemical information may be displayed in the form of images (e.g. as in FIG. 1) and/or in other forms such as Raman spectrograms (e.g. as in FIGS. 13, 14).
CARS signals can be detected with descanning (e.g. as in FIGS. 2, 4, 5 and 9-12) or without descanning (e.g. as in FIGS. 6-8). For single-band CARS signal detection, a single point detector can be used and descanning is not mandatory. However, for broadband signal detection, it is desirable to descan signals coming from different angles before the signals are dispersed by a spectrometer and/or to use a virtual slit spectrometer for which light entering the spectrometer at any point along a virtual slit is detected in the same manner (e.g. as in FIGS. 6-8).
White-light and CARS imaging may be performed on the same specimen (e.g. as in FIGS. 2 to 11) or on different but closely related specimens (e.g. as in FIG. 13).

Systems according to various example embodiments are illustrated in FIGS. 2 to 12. In FIGS. 3 to 12, elements which are the same as or can function in the same way as elements present in system 20-2 of FIG. 2 are identified with the same references as the elements of system 20-2.

FIG. 3 is a schematic view of an example system 20-3 configured in a way that allows forward detection of a CARS signal. Since a large portion of the CARS signal is emitted in the forward direction, embodiments which detect the CARS signal emitted in the forward direction can acquire stronger CARS signals, other factors being equal, than systems (e.g. as shown in FIG. 2) which detect CARS signals in the backward direction.

System 20-3 includes a second objective 20E on an opposite side of slide 21 from objective 22C. For white light imaging system 20-3 may operate in essentially the same manner as system 20-2 of FIG. 2. Trans-illumination unit 22B emits light which is delivered to illuminate slide 21 through objective 22C. A transmission light signal is collected by objective 22E reflected by flip mirror 22F and delivered to viewing system 23.

System 20-3 includes two flip mirrors, 20D and 20F which are both moved out of the light path to enable CARS imaging and are both positioned in the light path to enable white light imaging.

CARS light source 24 operates as described above with reference to FIG. 2. System 20-3 does not include an optical scanning unit 25. Scanning for CARS imaging may be achieved by moving slide 21 using stage 22A. A system like system 20-3 could be made to include a scanning unit before objective 22C and a descanning unit after objective 22E.

In system 20-3, the merged beam output by CARS light source 24 is expanded by a beam expander 22H to overfill the back aperture of objective 22C. The merged beam is focused by objective 22C onto slide 21. A forwardly-directed component of the CARS signal from the sample on slide 21 is collected by objective 22E and output from objective 22E in the form of collimated light which is delivered to a spectrometer 26 by way of one or more filters 22G that remove wavelengths corresponding to excitation beam 28 (i.e. remove wavelengths present in the merged beam delivered to the sample by objective 22C). Filter 22G may, for example be a short pass filter. In some embodiments, filter 22G comprises a long-pass filter 22G-1 which passes wavelengths corresponding to excitation beam 28 and reflects shorter wavelengths. The shorter wavelengths are then filtered by a short pass filter 22G-2. The combination of filters 22G-1 and 22G-2 may provide better rejection of excitation light 28 than either of filters 22G-1 and 22G-2 acting alone. The filtered CARS signal is delivered to a spectrometer 26 which operates as described above.

System 20-3 may be varied by exchanging the locations of trans-illumination unit 22B and viewing system 23.

FIG. 4 is a schematic illustration of an example system 20-4 which is similar in configuration to system 20-3 but includes optical scanning. System 20-4 includes a scanning unit 25-1 and a descanning unit 25-2. Scanning unit 25-1 operates to scan a spot at which excitation beam 28 is focused within a field of view on slide 21. Descanning unit 25-2 operates to collect a forward-directed component of the resulting CARS signal that originates from the location of the spot at which excitation beam 28 is focused. The operation of scanning unit 25-1 and descanning unit 25-2 are synchronized (mechanically and/or electronically) so that the location on slide 21 from which descanning unit 25-2 collects the CARS signal tracks the location of the spot at which scanning unit 25-1 focuses excitation beam 28.

Scanning unit 25-1 and descanning unit 25-2 may scan on one direction (e.g. an x- direction). Scanning in another direction (e.g. a y-direction) may be achieved by operating stage 22A to move slide 21 in the other direction. As compared to system 20-3, system 20-4 may provide increased imaging speed by providing faster scanning in one dimension than could readily be achieved using stage 22A.

Scanning unit 25-1 and descanning unit 25-2 may be implemented using any suitable optical scanning technology including those described elsewhere herein. System 20-4 includes rotating polygon scanners 25A-1 and 25A-2, for example.

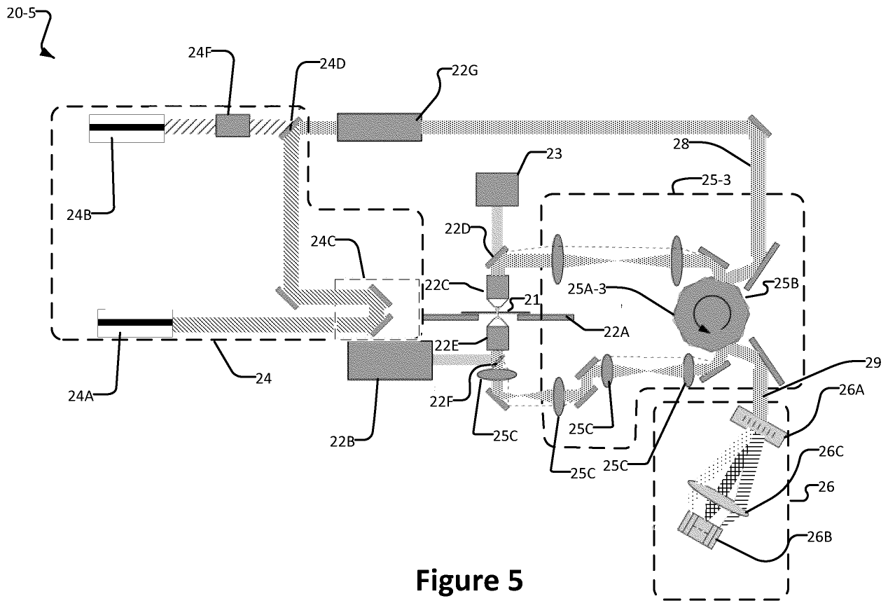

FIG. 5 is a schematic view of another example system 20-5 which is similar in concept to system 20-4 and includes a scanning unit 25-3 that combines the functionality of scanning unit 20-1 and descanning unit 25-2 of system 25-4. Scanning unit 25-3 includes a rotating member 25A-3 that includes reflecting facets 25B. Changing the angle of rotation of rotating member 25A-3 simultaneously performs scanning and descanning. In the illustrated embodiment, rotating member 25A-3 comprises a single rotating polygon scanner.

Excitation beam 28 is directed to be incident on rotating member 25-3A from a selected direction so that excitation beam 28 is reflected by facets 25B. As rotating member 25A-3 turns the angle on which excitation beam 28 enters objective 22C is varied, thus scanning the location of the spot at which excitation beam 28 is focused on slide 21 by objective 22C in an x-direction.

The CARS signal 29 collected by objective 22E is also guided to be incident on rotating member 25A-3 from a selected direction so that CARS signal 29 is reflected by facets 25B. As with other embodiments, suitable filters may be present in the optical path between objective 22E and spectrometer 26 to separate excitation light 28 from CARS signal 29. In the illustrated embodiment lenses 25C arranged to provide two lens pairs are arranged to direct the CARS signal onto rotating member 25A-3. CARS signal 29 is descanned by its interactions with facets 25B. Through this mechanism, scanning of excitation beam 28 and descanning of CARS signal 29 can be automatically and perfectly synchronized. The descanned CARS signals are stationary and are input into spectrometer 26.

System 20-5 may be configured for white-light imaging by positioning flip mirror 22F in the optical path and positioning flip mirror 22D out of the optical path. System 20-5 may be configured for CARS imaging by placing flip mirror 22D in the optical path and positioning flip mirror 22F out of the optical path.

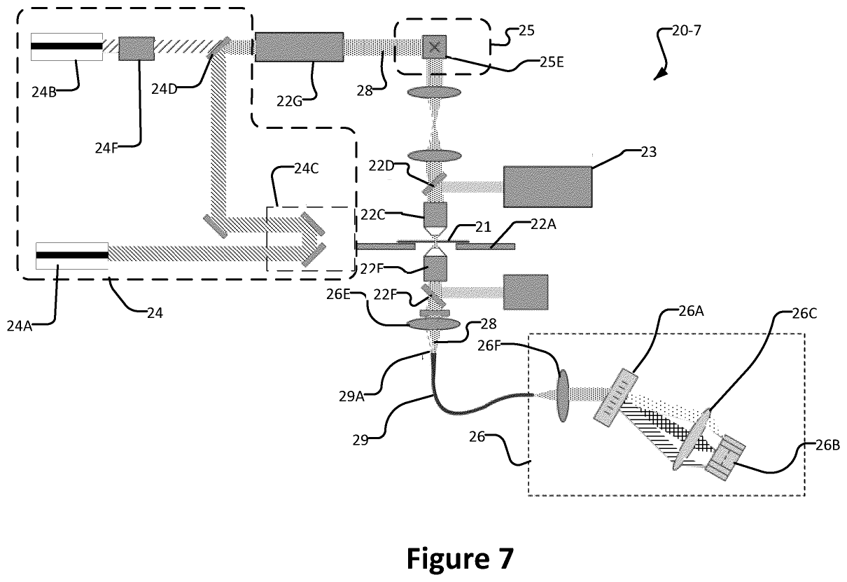

FIGS. 6 and 7 are schematic illustrations that respectively depict systems 20-6 and 20-7 in which light to be delivered to a spectrometer 26 is collected along a linear path, thereby facilitating collection of the CARS signal without descanning.

In system 20-6, spectrometer 26 is a virtual slit spectrometer which does not require a physical slit to define an entrance aperture. CARS signals collected by objective 22E are focused onto the virtual slit entrance aperture 26D of spectrometer 26, which is aligned with the direction in which the CARS signal is being scanned (e.g. the y-direction in FIG. 6). In system 20-6, lens 26E focuses the CARS signal into a spot that coincides with virtual slit 26D. Since excitation beam 28 is being scanned in the y-direction, the spot onto which lens 26E focuses the CARS signal will also be scanned along a line in the y-direction. System 20-6 may, for example, include a resonance scanner to scan excitation beam 28 in the y-direction.

In system 20-6 spectrometer 26 includes a light detecting array 26B in which each channel has a narrow width (e.g. 0.8 mm) in a direction perpendicular to the direction in which the CARS signal is being scanned and a relatively long length (e.g. 7 mm). The channels of light detecting array 26B are oriented so that their long dimension is aligned parallel to virtual slit 26D so that the CARS signal will be detected at all parts of the scanning cycle (i.e. regardless of the position of the spot at which the CARS signal is focused along virtual slit 26D). Operation of spectrometer 26 may be coordinated with the operation of scanner 25E so that separate spectra are obtained for different locations of the spot on the virtual slit onto which the CARS signal is focused. These different locations correspond to different locations (pixels) on slide 21.

System 20-7 is similar in concept to system 20-6. In system 20-7 the CARS signal is carried to spectrometer 26 by an optical element 29 which has a linear entrance aperture 29A and is configured to deliver light received at the entrance aperture to spectrometer 26. Linear entrance aperture 29A serves as a virtual slit entrance aperture for spectrometer 26.

Optical element 29 may, for example comprise an optical fiber bundle 29B. In such embodiments linear entrance aperture 29A may be provided at an entrance end of optical fiber bundle 29B by arranging individual optical fibers of fiber bundle 29B to form a linear array. For example, optical fiber bundle 29B may comprise a number of fibers (e.g. 100) that are arranged in a line in the x-direction so that signals from different scanning angles can all be collected by fibers of optical fiber bundle 29B.

In some embodiments optical element 29 comprises a fused fiber bundle or a light guide having rectangular-shaped ends.

The CARS signal is focused by lens 26E to a spot that lies on linear entrance aperture29A of optical element 29. As excitation beam 28 is scanned, the location of the spot is scanned back and forth along the linear entrance aperture of optical element 29. The exit end of optical element 29 emits light that has been collected anywhere along linear entrance aperture 29A at a spot or along a line that coincides with the location of an entrance aperture of spectrometer 26. The collected light is expanded to a beam by lens 26F before being separated by frequency by volume grating 26A.

In some embodiments the entrance aperture of spectrometer 26 comprises a virtual slit entrance aperture and the exit end of optical element 29 forms a line that is aligned to coincide with the virtual slit entrance aperture.

When a fiber bundle 29B or other optical element 29 is used to collect the CARS signal, there is a possibility that the efficiency with which the CARS signal is collected and detected by spectrometer 26 could vary with scanning angle (i.e. could vary with the position of the scanned spot along entrance aperture 29A of optical element 29. Where this is an issue, a calibration table or function may be applied to correct for the efficiency variation. A calibration table may, for example, be generated by measuring the scanning efficiency as a function of scanning angle or as a function of scanning angle and wavelength and generating correction factors that compensate for the efficiency variations. The measurements may, for example, comprise directing light of known intensity and wavelength spectrum into optical element 29 at different scanning angles and comparing the outputs of spectrometer 26 that correspond to the different scanning angles.

Apart from the inclusion of optical element 29, system 20-7 may be constructed like system 20-6.

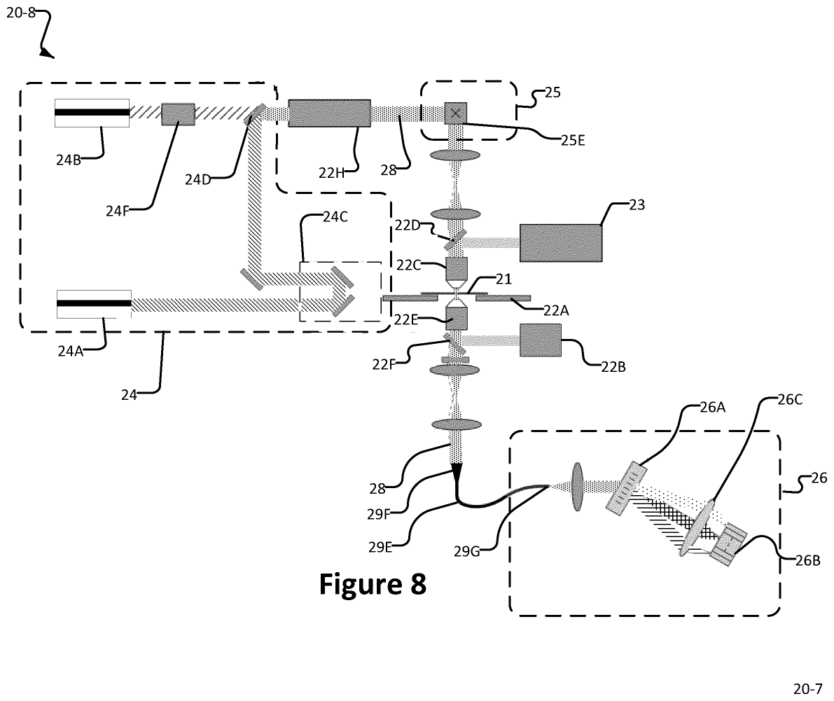

FIG. 8 is a schematic view of an example system 20-8 that is similar to systems 20-6 and 20-7 except for the design of the optical path by which the CARS signal is optically coupled into spectrometer 26. System 20-8 incorporates a tapered fiber/light guide 29E having an input end 29F that receives a CARS signal and an output end 29G that delivers the CARS signal into spectrometer 26. The CARS signal is presented at the location of input end 29F as a relatively large diameter beam (in a range of millimeters, e.g. 1 to 5 mm) that has essentially no lateral motion. Scanning causes small changes in the angle of the beam.

Tapered fiber/light guide 29E has a large core diameter at input end 29F and a significantly smaller core diameter at output end 29G.

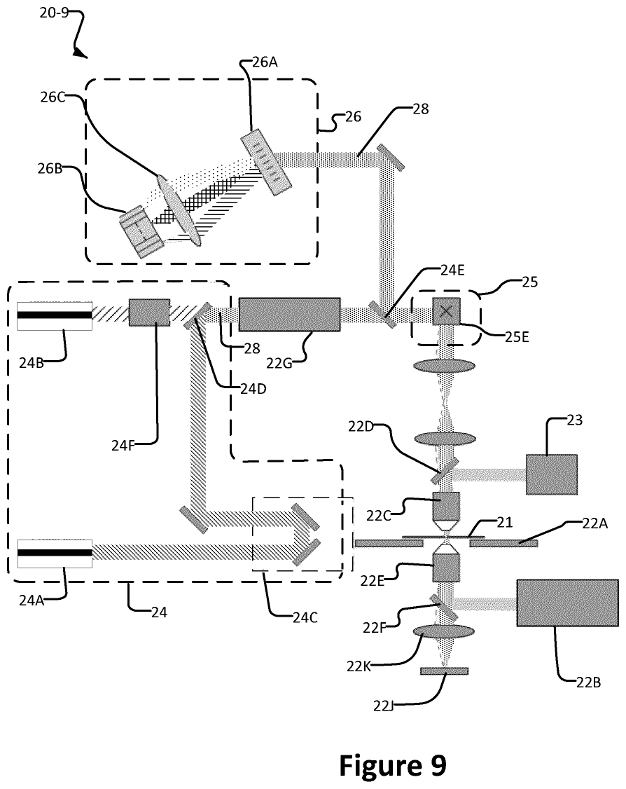

Systems according to some embodiments of the present technology are configurable for detection of CARS signal components that are emitted in both forward and backward directions. FIG. 9 shows an example system 20-9 that provides for combined backward and forward detection of CARS signals. In system 20-9, backward-emitted CARS signals are collected by objective 22C and delivered to scanner unit 25. Forward-emitted CARS signals are collected by objective 22E and directed onto a mirror 22J which reflects the forward-emitted CARS signals back through objective 22E, slide 21 and objective 22C. A lens 22K may be provided to focus the CARS signals onto mirror 22J.

Scanning unit 25 of system 20-9 may have any construction as described herein. For example, scanning unit 25 may include the combination of a resonance scanner and a galvo scanner or opto-acoustic scanner which respectively scan excitation beam 28 in the x and y directions.

Some embodiments of the present technology provide systems that facilitate imaging modes in addition to white-light and CARS imaging. FIG. 10 shows an example system 20-10 that includes various multimodal imaging channels. For white-light and CARS imaging, system 20-10 is configured and operated similarly to system 20-2 of FIG. 2. System 20-9 is additionally configured for confocal imaging of a sample on slide 21 using light from excitation beam 28 that is back-reflected at slide 21.

System 20-10 includes a polarizing beam splitter (PBS) 30A and a quarter-wave plate 30B in the optical path between excitation light source 24 and slide 21. Excitation beam 28 is polarized by PBS 30A in a first polarization state. Light from excitation beam 28 that is back reflected at slide 21 passes twice through quarter wave plate 30B and therefore has a second polarization state that is orthogonal to the first polarization state. The back-reflected light is directed by PBS 30A to detector 30C which may, for example, comprise an avalanche photodiode or other suitable light detector. Back-reflected light is focused by lens 30D onto a pinhole 30E located in front of light detector 30C for confocal detection.

System 20-10 (or any other system described herein) may additionally detect a second harmonic signal and/or two photo fluorescence signals at spectrometer 26. A second harmonic (SHG) signal and/or two-photon fluorescence (TPF) signal may be detected simultaneously with a CARS signal because the spectral components of these signals have wavelengths shorter than those of the CARS signal.

FIG. 11 is a schematic view of an example system 20-11 that is similar to system 20-10 but provides detectors 31A and 31B that respectively detect SHG and TPF signals and are separate from spectrometer 26 that detects CARS signals. In system 20-11 SHG and TPF signals are detected before descanning.

Backward-directed light is collected by objective 22C. A wavelength-selective mirror 31C directs longer wavelength light including the CARS signal to scanning unit 25 and directs shorter wavelength TPF and SHG signals toward detectors 31A and 31B. The SHG and TPF signals are separated by a longpass beamsplitter 31D that directs TPF signals that have longer wavelengths than SHG signals to detector 31B and directs SHG signals to detector 31A.

Example systems 20-2 to 20-11 perform CARS imaging using the intrapulse three-color excitation method. This method can be used for fingerprint CARS signal generation and fingerprint CARS imaging. Three-color excitation uses a broadband femtosecond laser to generate the pump and stokes beam and another narrow band laser to generate the probe beam. Bandwidth of the beam from the broadband laser should be broad enough that different wavelength components of laser pulses from the broadband laser work as pump and Stokes beams to cover vibration frequencies in the required fingerprint range. The resonance CARS signal can be extracted digitally from the raw spectra using the time-domain Kramers-Kronig transform. The present technology may also be applied to combine white light imaging with confocal Raman spectroscopy on the pre-selected region(s) of interest on a H&E stained slide.

FIG. 12 is a schematic illustration of an example system 20-12 that combines the functions of white light imaging and confocal Raman spectroscopy. System 20-12 is similar to system 20-2 except that light source 24 of system 20-12 includes a narrow bandwidth continuous-wave laser 24H which produces excitation beam 28. A laser line filter 24J cleans the spectrum of excitation beam 28. Excitation beam 28 passes through scanning unit 25 and objective 22C which focuses excitation beam 28 onto a the target point on H&E slide 21 to excite Raman signals. Backward-emitted Raman signals are collected by objective 22C, descanned by the scanning unit 25, and directed toward spectrometer 26 by filter 24K. The Raman signals are focused by a lens 26H into a pinhole 26J for confocal detection. After passing pinhole 26J, the Raman signals are collimated by lens 26K to form a beam that is dispersed by volume grating 26A. The dispersed Raman signals are detected by light detector array 26B to provide a Raman spectrum.

Example systems 20-2 to 20-12 perform white-light imaging and CARS imaging on the same slide 21. In some cases that may be undesirable or not workable (for example, where H&E stained slide 21 are prepared using procedures that leave the H&E stained slides contaminated with chemicals that interfere with CARS imaging). When direct CARS imaging/confocal Raman spectroscopy on the H&E stained slide is not possible or desirable, a minor modification of the pathology workflow can be applied to provide biochemical information regarding selected regions of the H&E stained slide using CARS and/or confocal Raman spectroscopy.

FIG. 13 is a schematic illustration of steps in an example method 130 in which CARS imaging and white light imaging are performed on two separate, but related slides. The workflow for use with systems 20-2 to 20-12 includes preparing two slides, 21A and 21B (Step S131). Slide 21A carries a tissue section 33A that is stained. Slide 21B carries a tissue section 33B that is adjacent to tissue section 33A and is unstained.

In step S132, slides 21A and 21B are both supported on stage 22A. In step 133 a mapping is created that indicates the locations of corresponding features that are shown in both of tissue sections 33A and 33B. Since sections 33A and 33B are adjacent sections each of the sections will, in general include the same structures. The mapping may, for example comprise a rotation and/or a translation which takes a point on section 33A to a corresponding point on section 33B. The mapping may, for example, be generated by processing low magnification white light images of sections 33A and 33B to locate coordinate pairs that indicate positions of structures present in both sections 33A and 33B. The processing may implement a coregistration method. A coordinate map resulting from the mapping provides correlations which relate the coordinates of any pixel on slide 21A to the coordinates of the same corresponding pixel (i.e. the pixel that coincided with the same structure) on slide 21B. These correlations can be used to find a region on slide 21B that corresponds to a ROI identified on slide 21A.

In step S134 a pathologist views H&E stained slide 21A. If the pathologist identifies finds any suspicious region(s), he/she can identify a ROI, e.g. by outlining the ROI using a suitable user interface (step S134). In step S135 the system scans to the location on slide 21B that corresponds to the ROI using the coordinate map. In step S136 a CARS image or confocal Raman spectra can be acquired from the ROI.

To facilitate method 130 it is useful for slide 21 to be operable to move slides 21A and 21B in x, y, and z directions as well as to rotate slides 21A and 21B in the x-y plane. Adjustment in the z direction may be used to focus imaging and excitation light on a stained tissue sample.

Method 130 may be performed using any of the systems as described herein. Another option is to perform method 130 in a system that is operable to simultaneously observe white light images of slide 21A and to perform CARS imaging on slide 21B. With such a system, CARS imaging of slide 21B may be performed while a pathologist is reviewing the white-light image of slide 21A. If the pathologist identifies a ROI on the white-light image then CARS imaging and/or confocal Raman spectroscopy of a region of slide 21B corresponding to the ROI may be performed while the pathologist continues to review the white light image. This approach can reduce or eliminate any need for the pathologist to wait for CARS imaging or confocal Raman spectroscopy of the ROI to be completed.

A system operable to simultaneously perform white-light imaging with CARS imaging and/or confocal Raman spectroscopy may, for example comprise a first system operable to perform white light imaging of a H&E slide 21A, a second system operable to perform CARS imaging and/or confocal Raman spectroscopy (these systems may or may not share some components) and a control system that includes a user interface operable to receive a definition of an ROI on slide 21A from a user, determine a mapping of the ROI onto slide 21B, and perform CARS imaging and/or confocal Raman spectroscopy of the ROI on slide 21B. In some embodiments the second system is operable in a white light imaging mode and the control system is operable to determine a relationship between the locations of corresponding structures in sections 33A and 33B by comparing white light images of slides 21A and 21B.

In all of the systems and methods described above, a white light imaging channel is provided for “gold standard” visual inspection of H&E stained slides. The visual inspection may be augmented by CARS and/or confocal Raman spectroscopy imaging of selected region(s) of interest (ROIs) in the field of view. For example, a pathologist may identify one or more ROIs by using a suitable interface to draw outline(s) around any suspicious region(s). The system may then acquire biochemical information for the selected ROI(s) to assist the diagnosis (e.g. by performing CARS imaging or confocal Raman spectroscopy). Scanning excitation beam 28 only in selected ROI(s) in the field of view of slide 21 speeds up the availability of biochemical information for the selected ROI(s) also avoids collection of irrelevant information. A ROI may, for example, include a bunch of cells, a single cell, or cytoplasm, or cell nucleus, etc.

Arbitrary Region of Interest (aROI) Imaging/Spectroscopy

Arbitrary region of interest CARS imaging/Raman spectroscopy allows a pathologist to acquire hyperspectral CARS images/Raman spectra only from selected ROI(s) (e.g. the suspicious regions of tissue). A ROI may have any desired shape such as, without limitation a point, line, circle, rectangle, or any other type of shape including a shape that lies within a freeform outline

As also discussed elsewhere herein one way for a user (e.g. a pathologist) to define a ROI is to mark a boundary of the ROI using a graphical user interface. The user interface may, for example, display a white light image that includes the ROI and may allow the user to draw an outline/semi-transparent shape that indicates the location of the ROI using a pointing device such as a pen, mouse, finger on a touch sensitive display, etc. over the region in the field of view of the white light imaging channel.

To realize this function, the coordinates and dimensions of the CARS imaging/confocal Raman spectroscopy channel are calibrated and co-registered with the coordinates of the white light imaging channel. After the calibration and co-registration has been performed excitation beam 28 may be directed to illuminate slide 21 at any point selected in the field of view of the white light imaging channel. A system as described herein may automatically scan the excitation beam to obtain a CARS image and/or confocal Raman spectrogram(s).

To reduce the time needed to obtain CARS imaging/confocal Raman spectroscopy for a ROI a system as described herein may be programmed/controlled to scan only within the ROI instead of a significantly larger pattern that includes the ROI. Scanning paths (made of points) are generated to evenly fill the ROI as defined by the user. For CARS imaging, at each point during the laser dwelling time, a CARS spectrum is acquired. After the CARS spectra from every point along the scanning paths are acquired, a hyperspectral CARS image of the ROI is generated, this image can be merged with the white light image to provide complementary molecular information. For confocal Raman spectroscopy, Raman signals over the whole ROI may be integrated to generate a single Raman spectrum.

The scanning path is advantageously as continuous as possible to avoid large-angle jumping of the scanners. A suitable scanning path may, for example, be generated for an arbitrarily shaped ROI using the following algorithm which decomposes the shape into a list of paths with each path as a closed-loop made of points.

Step 1, a map of the ROI is retrieved. The map may have the form of an image file. The image file may be generated in response to a user indicating a boundary of the ROI as described herein. The map may, for example, have the form of a digital mask with pixels belonging to the ROI having one value and pixels outside of the ROI having another value. In FIG. 14, pixels belonging to the ROI are shown as being black and pixels outside of the ROI are shown as being white.

Step 2, processes the map to identify vertices and edges of a boundary of the ROI. For step 2 the map is associated with a coordinate system so that the four corners of each pixel have corresponding integer coordinates. In this case, if p is a point specified by integer coordinates, point p will be surrounded by four pixels (assuming that the ROI is not at or overlapping with the edge of the map). If the four pixels have different colors, p is called a vertex. For any two vertexes, p and q, a directed edge pq can be defined if: the Euclidean distance between p and q is 1 and if the line p-q separates a black pixel from a white pixel. The direction of the edge may, for example be defined in such a way that when looking in the direction, the left side of the edge is a pixel that belongs to the ROI and the right side of the edge is a pixel that is outside of the ROI. In the alternative, the direction may be defined in the opposite way (i.e. so that when looking in the direction, the right side of the edge is a pixel that belongs to the ROI and the left side of the edge is a pixel that is outside of the ROI.

Step 3, applies a search pattern to find a first edge. For example, the search pattern may search the map for an edge from top to bottom and from left to right.

Step 4, identifies a closed path that begins and ends at the first edge. The closed path may be determined by starting with the first edge and sequentially finding next edges that extend the path. Traveling along the direction of a previously found edge (the first edge on the first iteration of Step 4), one will face four situations that will determine the next edge. These situations are illustrated in FIGS. 14A to 14D.

Step 4 is repeated to find edges sequentially until a closed path corresponding to the boundary of the ROI has been identified. A closed path has been formed when the end vertex of the last edge is the same as the start vertex of the first edge.

Step 5, The result of the repeated application of Step 4 is a list of edges. This list may be implemented as an ordered list of every vertex in the path. In Step 5 the map is modified by assigning to all of the pixels on the left side of every edge in the path the value associated with pixels that are outside of the ROI. This effectively implements an erosion operation on the map.

FIGS. 14E through 14G show stages in tracing a map and eroding the map as performed in Steps 4 and 5 for a very simple example ROI. In these Figures, black represents pixels that belong to a ROI and white represents pixels that are outside of the ROI. FIG. 14E shows an example ROI. The outline of the ROI is traced to generate a closed path. FIG. 15F shows the map after pixels on the left of the previously found path (outer path in FIG. 14E) are eroded (as indicated by filling with white color). The resulting modified map is traced to generate a second path. FIG. 14G shows the map after pixels on the left of the second path found shown in FIG. 14G are eroded (filled with white color). Since there are no more black pixels, the tracing process ends.

Step 6, repeats steps 3-5 until the map is empty (filled with pixels for which the pixel values represent pixels outside of the ROI).

In Step 7, all of the paths generated by the process of Step 4 are linked and converted to a list of pixel coordinates and corresponding driving values for a scanning unit. The driving values may, for example comprise voltages for driving galvo or other scanners to direct excitation light 28 to locations on a slide 21 that correspond to the pixel coordinates.

The tracing density produced by the above algorithm may be changed by upsampling or downsampling the map. For example, pixel binning may be used to downsample the map to reduce the tracing density (make paths more widely spaced apart). For example, if a map is provided in the form of an image having a size of 512×512 pixels, the tracing density may be reduced by half by performing 2×2 pixel binning on the map to result in a new map in the form of an image having a size of 256×256 pixels. The above algorithm may be performed using the reduced-size map. After tracing paths have been determined as described above the coordinates of every vertex in the tracing path may be multiplied by 2 to provide a tracing path that covers the ROI at a reduced density. This makes the tracing process faster and the scanning time shorter while ensuring that the ROI is fully covered by excitation beam 28.

The above algorithm is derived from the path-decomposition part of the Portrace algorithm which is described in [15]. This tracing algorithm is a kind of parallel contour path generation process. The algorithm can be used to establish a continuous path for scanning an arbitrarily shaped ROI to a large extent. In some cases, depending on the shape of the ROI and the tracing density some disconnected residual pixels may remain. Scanning these disconnected pixels might not be practical without introducing discontinuities, which is undesirable. In many cases the number of such pixels is small and this problem can be solved by ignoring paths that include fewer than some threshold number of edges. For further improvement, a more advanced path tracing algorithm could be used.

### Example Controllers

Systems as described herein can include controllers that provide functionality such as:
receiving user input defining ROIs,
generating control signals to operate scanning units;
determine correspondences between point(s) on a slide 21 identified in white light images and control signals for a scanning unit which will cause the scanning unit to deliver an excitation beam 28 to the same point(s) on the slide 21;
determining paths for scanning a particular ROI;
controlling a light detector array to obtain spectra in coordination with scanning;
storing images, confocal Raman spectra and/or CARS images;
automatically identifying or identifying and removing components of a Raman spectrum or CARS image that correspond to one or more residual processing chemicals and/or stains;
processing a Raman signal or CARS image to identify biochemicals present in a tissue sample;
preparing images that indicate tissue characteristics derived from processing Raman signals and/or CARS images (e.g. DNA concentration, concentration(s) of one or more biochemicals in a tissue sample etc.).

In some embodiments such controllers are implemented using one or more of: specifically designed hardware, configurable hardware, programmable data processors configured by the provision of software (which may optionally comprise “firmware”) capable of executing on the data processors, special purpose computers or data processors. Such controllers may be specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

In some embodiments a controller has access to a data store that contains data that characterizes Raman spectra of chemicals that may be used to process a histopathology slide (e.g. formaldehyde, xylene, paraffin) and/or one or more stains (e.g. hematoxylin, eosin, Papanicolaou stain or its components, Masson's Trichrome stain or its components). The processor may use this stored data to identify component(s) of a Raman or CARS signal that correspond to processing chemicals and/or stains. The processor may use this information to remove components of the Raman or CARS signal that correspond to processing chemicals and/or stains.

In some embodiments the processor is configured to receive input data that characterizes the preparation of a slide. The input data may, for example, directly or indirectly identify processing chemicals and/or stains used in preparation of the slide. The processor may use the input information to assist in identifying and/or removing from a Raman or CARS signal components that correspond to one or more processing chemicals and/or stains that may be present in the slide.

Some aspects of the present invention comprise or consist of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

### Example Applications

An example of the application of the methods and apparatus described herein is to help visualize DNA content of cells on a histology slide (e.g. a H&E stained slide). The cell nucleus has multiple Raman peaks in the fingerprint region including 785 cm⁻¹, 1095 cm⁻¹, 1487 cm⁻¹, and 1578 cm⁻¹ [16]. Here we take 785 cm⁻¹ as an example. To generate a CARS signal in this Raman peak, one can employ narrowband CARS by use of a 785 nm pump laser and an 836 nm Stokes laser. The corresponding anti-Stokes signal has a center wavelength of 739 nm. After the H&E slide is viewed by a pathologist, he/she can select (e.g. by outlining) a region of interest for CARS imaging. The resulting CARS image will show the signal intensity distribution of the 785 cm⁻¹ peak in the selected region, which reflects the DNA quantity and distribution. CARS mages that show the distributions of other biochemical that have discernable Raman peaks may be prepared in an analogous manner. The same DNA CARS signal can also be obtained using the above-mentioned three-color excited broadband CARS imaging as illustrated in various embodiments.

It is also worth mentioning that confocal Raman spectroscopy although being very slow to get useful information for the whole slide, could be practical and helpful by providing local biomolecule information from a pre-selected small region guided by H&E histopathology.

While particular elements, embodiments and applications of the present disclosure have been shown and described, it will be understood, that the scope of the disclosure is not limited thereto, since modifications can be made by those skilled in the art without departing from the scope of the present disclosure, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and sub-combinations are intended to fall within the scope of this disclosure.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Where a component (e.g. a lens, filter, mirror, objective, scanner, stage, controller, processor, assembly, device, light sensor etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

### Interpretation of Terms

Unless the context clearly requires otherwise, throughout the description and the claims:
“comprise”, “comprising”, “including” and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

Reference to “some embodiments,” “an embodiment,” or the like, means that a particular feature, structure, step, process, or characteristic described in connection with the embodiment is included in at least one embodiment. Thus, the phrases “in some embodiments,” “in an embodiment,” or the like, throughout this disclosure are not necessarily all referring to the same embodiment and may refer to one or more of the same or different embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

### REFERENCES

1. C. M. Krishna, G. Sockalingum, L. Venteo, R. A. Bhat, P. Kushtagi, M. Pluot, and M. Manfait, “Evaluation of the suitability of ex vivo handled ovarian tissues for optical diagnosis by Raman microspectroscopy,” Biopolymers: Original Research on Biomolecules 79, 269-276 (2005).
2. R. Galli, O. Uckermann, E. Koch, G. Schackert, M. Kirsch, and G. Steiner, “Effects of tissue fixation on coherent anti-Stokes Raman scattering images of brain,” J Biomed Opt 19, 071402 (2014).
3. G. de Vito, P. Parlanti, R. Cecchi, S. Luin, V. Cappello, I. Tonazzini, and V. Piazza, “Effects of fixatives on myelin molecular order probed with RP-CARS microscopy,” Appl Opt 59, 1756-1762 (2020).
4. E. Ó Faoláin, M. B. Hunter, J. M. Byrne, P. Kelehan, M. McNamara, H. J. Byrne, and F. M. Lyng, “A study examining the effects of tissue processing on human tissue sections using vibrational spectroscopy,” Vib Spectrosc 38, 121-127 (2005).
5. S. A. Mian, H. E. Colley, M. H. Thornhill, and I. u. Rehman, “Development of a Dewaxing Protocol for Tissue-Engineered Models of the Oral Mucosa Used for Raman Spectroscopic Analysis,” Appl Spectrosc Rev 49, 614-617 (2014).
6. A. Downes, “Raman Microscopy and Associated Techniques for Label-Free Imaging of Cancer Tissue,” Appl Spectrosc Rev 50, 641-653 (2015).
7. L. Andronia, V. Miresan, A. Coroian, I. Pop, C. Răducu, A. Rotaru, D. Cocan, S. C. Pánzaru, I. Domsa, and C. O. Coroian, “Raman Spectroscopy of the Hematoxylin-Eosin Stained Tissue,” ProEnvironment Promediu 8 (2015).
8. M. D. Morris, N. J. Crane, L. E. Gomez, and M. A. Ignelzi, Jr., “Compatibility of staining protocols for bone tissue with Raman imaging,” Calcif Tissue Int 74, 86-94 (2004).
9. J.-x. Cheng, A. Volkmer, L. D. Book, and X. S. Xie, “An epi-detected coherent anti-Stokes Raman scattering (E-CARS) microscope with high spectral resolution and high sensitivity,” The Journal of Physical Chemistry B 105, 1277-1280 (2001).
10. J.-x. Cheng, A. Volkmer, L. D. Book, and X. S. Xie, “Multiplex coherent anti-Stokes Raman scattering microspectroscopy and study of lipid vesicles,” The Journal of Physical Chemistry B 106, 8493-8498 (2002).
11. M. Müller, and J. M. Schins, “Imaging the thermodynamic state of lipid membranes with multiplex CARS microscopy,” The Journal of Physical Chemistry B 106, 3715-3723 (2002).
12. G. W. Wurpel, J. M. Schins, and M. MOller, “Chemical specificity in three-dimensional imaging with multiplex coherent anti-Stokes Raman scattering microscopy,” Opt Lett 27, 1093-1095 (2002).
13. J.-X. Cheng, and X. S. Xie, “Coherent anti-Stokes Raman scattering microscopy: instrumentation, theory, and applications,” The Journal of Physical Chemistry B 108, 827-840 (2004).
14. C. H. Camp Jr, Y. J. Lee, J. M. Heddleston, C. M. Hartshorn, A. R. H. Walker, J. N. Rich, J. D. Lathia, and M. T. Cicerone, “High-speed coherent Raman fingerprint imaging of biological tissues,” Nature photonics 8, 627-634 (2014).
15. P. Selinger, “Potrace: a polygon-based tracing algorithm,” Potrace (online), http://potrace.sourceforge.net/potrace.pdf (2009-07-01) (2003).
16. T. Guerenne-Del Ben, Z. Rajaofara, V. Couderc, V. Sol, H. Kano, P. Leproux, and J. M. Petit, “Multiplex coherent anti-Stokes Raman scattering highlights state of chromatin condensation in CH region,” Sci Rep 9, 13862 (2019).

1. A system for multimodal imaging of histology-stained slides, the system comprising:
a white light imaging system configured to provide a white light image of a field of view of a tissue sample on the histology-stained slide;
a user interface operable to receive user input specifying a region of interest (ROI) on the tissue sample;
a Raman spectroscopy system comprising a light source operable to generate an excitation beam, and a scanner operable to scan the excitation beam over the ROI, and a spectrometer coupled to receive Raman scattered light resulting from interaction of the excitation beam and the tissue sample; the Raman spectroscopy system configured to perform Raman spectroscopy on the ROI to obtain Raman spectra; and
a display connected to display a Raman spectrogram and/or an image of the ROI acquired by the Raman spectroscopy system.

2.-3. (canceled)

4. The system according to claim 1 wherein the Raman spectroscopy system comprises a coherent anti-Stokes Raman scattering (CARS) system configured to generate a hyperspectral or multispectral Raman image of at least the ROI and the spectrometer is integrated with the CARS system.

5.-11. (canceled)

12. The system according to claim 1 wherein the spectrometer is a virtual slit spectrometer and the scanner causes the Raman scattered light to be scanned back and forth along a virtual slit of the spectrometer.

13. (canceled)

14. The system according to claim 1 wherein the Raman spectroscopy system is arranged to receive the Raman scattered light that is emitted in a forward direction on a side of the histology stained slide opposite to a side of the histology stained slide on which the excitation beam is incident.

15. (canceled)

16. The system according to claim 14 wherein the scanner comprises a first rotating polygonal mirror and the Raman scattered light is directed to be reflected from the first rotating polygonal mirror and to be descanned by the first rotating polygonal mirror.

17. The system according to claim 14 wherein the scanner comprises a first rotating polygonal mirror and the system comprises a second rotating polygonal mirror, wherein the Raman scattered light is directed to be reflected from the second rotating polygonal mirror and to be descanned by the second rotating polygonal mirror.

18. The system according to claim 1 wherein the Raman spectroscopy system is arranged to receive the Raman scattered light that is emitted in a backward direction on a side of the histology stained slide that is the same as a side of the histology stained slide on which the excitation beam is incident.

19. The system according to claim 18 wherein the Raman scattered light is guided to pass through the scanner and to be descanned by the scanner.

20. (canceled)

21. The system according to claim 1 wherein the system is configured to collect: a forward component of the Raman scattered light that is emitted in a forward direction on a side of the histology stained slide opposite to a side of the histology stained slide on which the excitation beam is incident, and a backward component of the Raman scattered light that is emitted on a side of the histology stained slide that is the same as a side of the histology stained slide on which the excitation beam is incident, to combine the forward and backward components of the Raman scattered light and to direct the combined forward and backward components of the Raman scattered light into the spectrometer and the system comprises an optical reflector arranged to redirect one of the forward and backward components of the Raman scattered light to pass though the histology stained slide before reaching the spectrometer.

22. (canceled)

23. The system according to claim 1 comprising a controller configured to obtain spectra from the spectrometer in coordination with operation of the scanner and to associate each of the spectra with a location within the ROI wherein the controller comprises a data store containing process information that characterizes Raman spectra of processing chemicals and/or stains and the controller is configured to identify one or more components of the obtained spectra of the Raman scattered light that correspond to the processing chemicals and/or stains based on the process information.

24. The system according to claim 23 wherein the controller is configured to remove the components of the obtained spectra of the Raman scattered light that correspond to the processing chemicals and/or stains.

25. (canceled)

26. The system according to claim 1 comprising a controller configured to obtain spectra from the spectrometer in coordination with operation of the scanner and to associate each of the spectra with a location within the ROI, wherein the tissue sample comprises a first part and a second part, the first part of the tissue sample being on the histology stained slide and the second part of the tissue sample being on a second slide wherein the white light imaging system is configured to image the first part of the tissue sample on the histology stained slide, the Raman spectroscopy system is configured to obtain the Raman spectrogram and/or image from the second part of the tissue sample and the controller is configured to create a mapping that indicates the locations of corresponding features that are shown in both of the first and second parts of the tissue sample and the mapping comprises a transformation comprising a rotation and/or a translation which takes a point on the first part of the tissue sample to a corresponding point on the second part of the tissue sample.

27. The system according to claim 1 comprising a controller configured to obtain spectra from the spectrometer in coordination with operation of the scanner and to associate each of the spectra with a location within the ROI, wherein the tissue sample comprises a first part and a second part, the first part of the tissue sample being on the histology stained slide and the second part of the tissue sample being on a second slide wherein the white light imaging system is configured to image the first part of the tissue sample on the histology stained slide, the Raman spectroscopy system is configured to obtain the Raman spectrogram and/or image from the second part of the tissue sample, the controller is configured to create a mapping that indicates the locations of corresponding features that are shown in both of the first and second parts of the tissue sample and the controller is configured to generate the mapping by processing low magnification white light images of the first and second parts of the tissue sample to locate coordinate pairs that indicate positions of structures present in both of the first and second parts of the tissue sample.

28. The system according to claim 1 wherein the user interface comprises a pointing device operable to specify the ROI by drawing a boundary of the ROI on the white light image.

29.-32. (canceled)

33. A method for multimodal imaging of a histology-stained slide, the method comprising:
imaging the histology stained slide with a white light imaging system to provide a white light image of a field of view of a tissue sample on the histology-stained slide and displaying the white light image to a user;
receiving by a user interface user input specifying a region of interest (ROI) on the tissue sample;
based on the user input, operating a Raman spectroscopy system to obtain Raman spectra for a portion of the tissue sample corresponding to the ROI; and,
displaying a Raman spectrogram and/or an image of the ROI acquired by the Raman spectroscopy system.

34.-40. (canceled)

41. The method according to claim 33 comprising scanning the Raman scattered light back and forth along a virtual slit of a virtual slit spectrometer.

42.-48. (canceled)

49. The method according to claim 33 comprising creating a mapping that indicates the locations of corresponding features that are shown in both of the first and second parts of the tissue sample, wherein the mapping comprises a transformation comprising a rotation and/or a translation which takes a point on the first part of the tissue sample to a corresponding point on the second part of the tissue sample.

50. The method according to claim 33 comprising creating a mapping that indicates the locations of corresponding features that are shown in both of the first and second parts of the tissue sample, wherein creating the mapping comprises processing low magnification white light images of the first and second parts of the tissue sample to locate coordinate pairs that indicate positions of structures present in both of the first and second parts of the tissue sample.

51. The method according to claim 33 comprising specifying the ROI by drawing a boundary of the ROI on the white light image.

52. The method according to claim 33 comprising, without removing the histology stained slide from an apparatus comprising the white light imaging system and the Raman spectroscopy system, imaging the tissue sample using one or more additional imaging modality, the additional imaging modality selected from the group consisting of: reflectance confocal imaging; two-photon fluorescence imaging; and second harmonic generation imaging.

53.-55. (canceled)
